(19) United States
(12) Patent Application Publication
(10) Pub. No.: US 2012/0101185 A1
(43) Pub. Date: Apr. 26, 2012

(54) POLYMERIC MATERIALS COMPRISING BARIUM SULPHATE

(75) Inventors: Craig Valentine, Lancashire (GB); Andrew Elleray, Lancashire (GB); Keith Cartwright, Lancashire (GB)

(73) Assignee: INVIBIO LIMITED, Lancashire (GB)

(21) Appl. No.: 13/265,601

(22) PCT Filed: Apr. 16, 2010
(86) PCT No.: PCT/GB10/50631

§ 371 Date: Jan. 6, 2012

(30) Foreign Application Priority Data
Apr. 21, 2009 (GB) 0906823.0

(51) Int. Cl. A61F 2/02; C08L 61/16; C08K 3/30

(52) U.S. Cl. 523/113; 524/423

(57) ABSTRACT

The use of barium sulphate for increasing the impact strength of a polyaryletherketone and/or a polyarylethersulphone involves compounding the barium sulphate with the polymeric material and including water in the composition prepared. The material may be used in implantable prostheses.

This invention relates to polymeric materials and particularly, although not exclusively, relates to polymeric materials with improved Notched Izod Impact Strength.

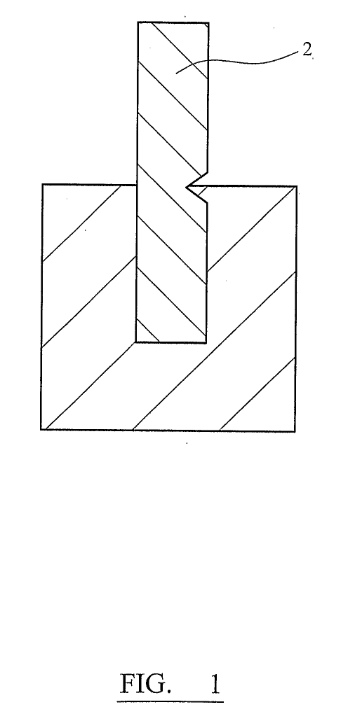

Notched Izod Impact Strength (NIIS) may be assessed in accordance with ISO180 wherein a specimen 2 as shown in FIG. 1 is held as a vertical cantilevered beam and is broken by a pendulum. Impact occurs on the notched side of the specimen.

The NIIS test may be of use in assessing the strength of parts made from polymeric materials which have a notch (or a shape similar to a notch) as part of the design or may be of use in assessing the impact on the strength of a part by damage which may produce a notch (or a shape similar to a notch) in the part.

In the field of orthopaedic implants, the notch sensitivity is, in some cases, a potential cause of failure of an implant. For example, dynamic stabilization rods may be pre-notched during manufacture or may be notched during the surgical procedure. The notched areas have been identified as possible failure points. Similarly, in hip joints, there is potential for acetabular cup impingement during implantation which could result in notching and the risk of fracture. In general terms, notch sensitivity may be important in sports medicine applications (e.g. for suture anchors and screws), in trauma (e.g. for plates, nails and screws) and in orthopaedics (e.g. for acetabular cups, femoral heads and humoral heads).

In many non-medical applications NIIS is important, particularly if there is a risk that a part may become damaged so that a notch is defined.

It is an object of the present invention to address the above described problems.

According to a first aspect of the invention, there is provided the use of barium sulphate for increasing the impact strength of a polymeric material which has a moiety of formula

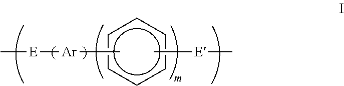

and/or a moiety of formula

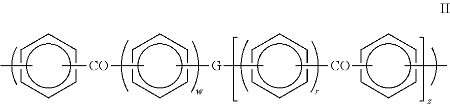

and/or a moiety of formula

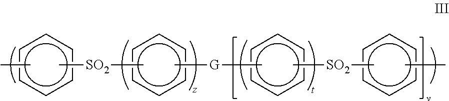

wherein m, r, s, t, v, w and z independently represent zero or a positive integer, E and E′ independently represent an oxygen or a sulphur atom or a direct link, G represents an oxygen or sulphur atom, a direct link or a —O—Ph—O— moiety where Ph represents a phenyl group and Ar is selected from one of the following moieties (i)**, (i) to (iv) which is bonded via one or more of its phenyl moieties to adjacent moieties

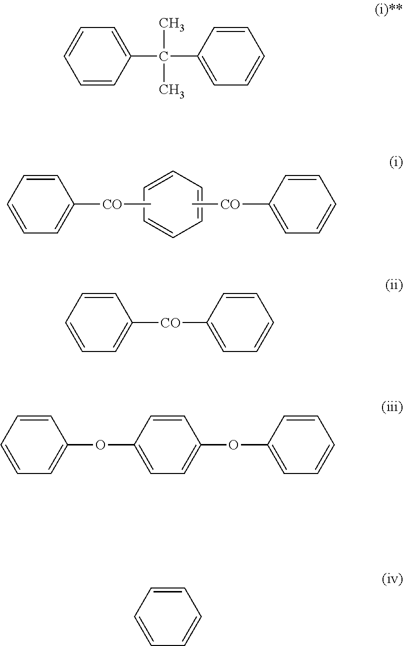

Unless otherwise stated in this specification, a phenyl moiety has 1,4-, linkages to moieties to which it is bonded.

In (i), the middle phenyl may be 1,4- or 1,3-substituted. It is preferably 1,4-substituted.

Said polymeric material may include more than one different type of repeat unit of formula I; and more than one different type of repeat unit of formula II; and more than one different type of repeat unit of formula III. Preferably, however, only one type of repeat unit of formula I, II and/or III is provided.

Said moieties I, II and III are suitably repeat units. In the polymeric material, units I, II and/or III are suitably bonded to one another—that is, with no other atoms or groups being bonded between units I, II and III.

Phenyl moieties in units I, II and III are preferably not substituted. Said phenyl moieties are preferably not cross-linked.

Where w and/or z is/are greater than zero, the respective phenylene moieties may independently have 1,4- or 1,3-linkages to the other moieties in the repeat units of formulae II and/or III. Preferably, said phenylene moieties have 1,4-linkages.

Preferably, the polymeric chain of the polymeric material does not include a —S— moiety. Preferably, G represents a direct link.

Suitably, “a” represents the mole % of units of formula I in said polymeric material, suitably wherein each unit I is the same; “b” represents the mole % of units of formula II in said polymeric material, suitably wherein each unit II is the same; and “c” represents the mole % of units of formula III in said polymeric material, suitably wherein each unit III is the same. Preferably, a is in the range 45-100, more preferably in the range 45-55, especially in the range 48-52. Preferably, the sum of b and c is in the range 0-55, more preferably in the range 45-55, especially in the range 48-52. Preferably, the ratio of a to the sum of b and c is in the range 0.9 to 1.1 and, more preferably, is about 1. Suitably, the sum of a, b and c is at least 90, preferably at least 95, more preferably at least 99, especially about 100. Preferably, said polymeric material consists essentially of moieties I, II and/or III.

Said polymeric material may be a homopolymer having a repeat unit of general formula

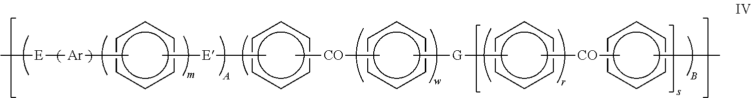

or a homopolymer having a repeat unit of general formula

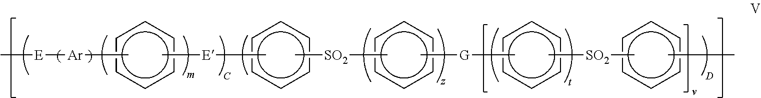

or a random or block copolymer of at least two different units of IV and/or V
wherein A, B, C and D independently represent 0 or 1 and E, E′, G, Ar, m, r, s, t, v, w and z are as described in any statement herein.

Preferably, m is in the range 0-3, more preferably 0-2, especially 0-1. Preferably, r is in the range 0-3, more preferably 0-2, especially 0-1. Preferably t is in the range 0-3, more preferably 0-2, especially 0-1. Preferably, v is 0 or 1. Preferably v is 0 or 1. Preferably, w is 0 or 1. Preferably z is 0 or 1.

Preferably, said polymeric material is a homopolymer having a repeat unit of general formula IV.

Preferably Ar is selected from the following moieties (xi)** and (vii) to (x)

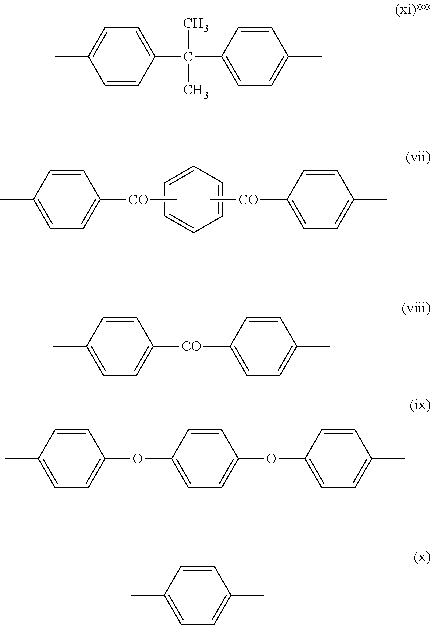

In (vii), the middle phenyl may be 1,4- or 1,3-substituted. It is preferably 1,4-substituted.

Suitable moieties Ar are moieties (i), (ii), (iii) and (iv) and, of these, moieties (i), (ii) and (iv) are preferred. Other preferred moieties Ar are moieties (vii), (viii), (ix) and (x) and, of these, moieties (vii), (viii) and (x) are especially preferred.

An especially preferred class of polymeric materials are polymers (or copolymers) which consist essentially of phenyl moieties in conjunction with ketone and/or ether moieties. That is, in the preferred class, said polymeric material does not include repeat units which include —S—, —SO₂— or aromatic groups other than phenyl. Preferred polymeric materials of the type described include:
(a) a polymeric material consisting essentially of units of formula IV wherein Ar represents moiety (iv), E and E′ represent oxygen atoms, m represents 0, w represents 1, G represents a direct link, s represents 0, and A and B represent 1 (i.e. polyetheretherketone).
(b) a polymeric material consisting essentially of units of formula IV wherein E represents an oxygen atom, E′ represents a direct link, Ar represents a moiety of structure (i), m represents 0, A represents 1, B represents 0 (i.e. polyetherketone);
(c) a polymeric material consisting essentially of units of formula IV wherein E represents an oxygen atom, Ar represents moiety (i), m represents 0, E′ represents a direct link, A represents 1, B represents 0, (i.e. polyetherketoneketone).
(d) a polymeric material consisting essentially of units of formula IV wherein Ar represents moiety (i), E and E′ represent oxygen atoms, G represents a direct link, m represents 0, w represents 1, r represents 0, s represents 1 and A and B represent 1. (i.e. polyetherketoneetherketoneketone).
(e) a polymeric material consisting essentially of units of formula IV, wherein Ar represents moiety (iv), E and E′ represents oxygen atoms, G represents a direct link, m represents 0, w represents 0, s, r, A and B represent 1 (i.e. polyetheretherketoneketone).
(f) a polymeric material comprising units of formula IV, wherein Ar represents moiety (iv), E and E′ represent oxygen atoms, m represents 1, w represents 1, A represents 1, B represents 1, r and s represent 0 and G represents a direct link (i.e. polyether-diphenyl-ether-phenyl-ketone-phenyl-).

Said polymeric material may be amorphous or semi-crystalline. Said polymeric material is preferably semi-crystalline. The level and extent of crystallinity in a polymer is preferably measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning Calerimetry (DSC).

The level of crystallinity in said polymeric material may be at least 1%, suitably at least 3%, preferably at least 5% and more preferably at least 10%. In especially preferred embodiments, the crystallinity may be greater than 30%, more preferably greater than 40%, especially greater than 45%.

The main peak of the melting endotherm (Tm) for said polymeric material (if crystalline) may be at least 300° C.

Said polymeric material may consist essentially of one of units (a) to (f) defined above.

Said polymeric material preferably comprises, more preferably consists essentially of, a repeat unit of formula (XX)

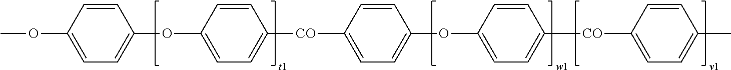

where t1, and w1 independently represent 0 or 1 and v1 represents 0, 1 or 2. Preferred polymeric materials have a said repeat unit wherein t1=1, v1=0 and w1=0; t1=0, v1=0 and w1=0; t1=0, w1=1, v1=2; or t1=0, v1=1 and w1=0. More preferred have t1=1, v1=0 and w1=0; or t1=0, v1=0 and w1=0. The most preferred has t1=1, v1=0 and w1=0.

In preferred embodiments, said polymeric material is selected from polyetheretherketone, polyetherketone, polyetherketoneetherketoneketone and polyetherketoneketone. In a more preferred embodiment, said polymeric material is selected from polyetherketone and polyetheretherketone. In an especially preferred embodiment, said polymeric material is polyetheretherketone.

Said polymeric material may have a Notched Izod Impact Strength (specimen 80 mm×10 mm×4 mm with a cut 0.25 mm notch (Type A), tested at 23° C., in accordance with ISO180) (hereinafter referred to as NIIS) of at least 4 KJm⁻², preferably at least 5 KJm⁻², more preferably at least 6 KJm⁻². Said Notched Izod Impact Strength, measured as aforesaid, may be less than 10 KJm⁻², suitably less than 8 KJm⁻².

NIIS hereinafter referred to is measured as aforesaid, unless otherwise stated.

Said polymeric material suitably has a melt viscosity (MV) of at least 0.06 kNsm⁻², preferably has a MV of at least 0.085 kNsm⁻², more preferably at least 0.12 kNsm⁻², especially at least 0.14 kNsm⁻².

MV is suitably measured using capillary rheometry operating at 400° C. at a shear rate of 1000 s⁻¹ using a tungsten carbide die, 0.5×3.175 mm.

Said polymeric material may have a MV of less than 1.00 kNsm⁻², preferably less than 0.5 kNsm⁻².

Said polymeric material may have a MV in the range 0.09 to 0.5 kNsm⁻², preferably in the range 0.14 to 0.5 kNsm⁻².

Said polymeric material may have a tensile strength, measured in accordance with ISO527 (specimen type 1b) tested at 23° C. at a rate of 50 mm/minute of at least 20 MPa, preferably at least 60 MPa, more preferably at least 80 MPa. The tensile strength is preferably in the range 80-110 MPa, more preferably in the range 80-100 MPa.

Said polymeric material may have a flexural strength, measured in accordance with ISO178 (80 mm×10 mm×4 mm specimen, tested in three-point-bend at 23° C. at a rate of 2 mm/minute) of at least 50 MPa, preferably at least 100 MPa, more preferably at least 145 MPa. The flexural strength is preferably in the range 145-180 MPa, more preferably in the range 145-164 MPa.

Said polymeric material may have a flexural modulus, measured in accordance with ISO178 (80 mm×10 mm×4 mm specimen, tested in three-point-bend at 23° C. at a rate of 2 mm/minute) of at least 1 GPa, suitably at least 2 GPa, preferably at least 3 GPa, more preferably at least 3.5 GPa. The flexural modulus is preferably in the range 3.5-4.5 GPa, more preferably in the range 3.5-4.1 GPa.

For the avoidance of doubt, the aforesaid characteristics of said polymeric material refer to polymeric material per se (ie unfilled).

Said polymeric material may be amorphous or semi-crystalline. It is preferably semi-crystalline.

The level and extent of crystallinity in a polymer is preferably measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning Calerimetry (DSC).

The level of crystallinity of said polymeric material may be at least 1%, suitably at least 3%, preferably at least 5% and more preferably at least 10%. In especially preferred embodiments, the crystallinity may be greater than 25%.

The main peak of the melting endotherm (Tm) of said polymeric material (if crystalline) may be at least 300° C.

Said barium sulphate may have a D₁₀ particle size in the range 0.1 to 1.0 μm; a D₅₀ particle size suitably in the range 0.5 μm to 2 μm and a D₉₀ particle size suitably in the range 1.0 μm to 5 μm. The D₁₀ may be in the range 0.1 to 0.6 μm, preferably 0.2 to 0.5 μm. The D₅₀ may be in the range 0.7 μm to 1.5 μm, preferably 0.8 to 1.3 μm. The D₉₀ may be in the range 1.5 to 3 μm, preferably in the range 2.0 to 2.5 μm.

The ratio of the wt % of barium sulphate to the wt % of said polymeric material may be greater than 0.04, is suitably greater than 0.07, is preferably greater than 0.10, is more preferably greater than 0.13, and is especially greater than 0.16. In some cases, said ratio may be greater than 0.20 or 0.22. The ratio may be less than 0.4, or less than 0.3.

Said barium sulphate is preferably intimately mixed with the polymeric material, suitably so the barium sulphate and polymeric material define a substantially homogenous mixture.

The wt % of barium sulphate in a composition which includes barium sulphate and said polymeric material may be at least 5 wt %, suitably at least 8 wt %, preferably at least 10 wt %, more preferably at least 13 wt %, especially at least 16 wt %. Said wt % of barium sulphate may be less than 30 wt % or less than 25 wt %. The wt % of said polymeric material in a composition which includes barium sulphate (and, optionally water as hereinafter described) may be at least 40 wt %, preferably at least 45 wt %, more preferably at least 50 wt %. In some cases, greater than 99 wt % of said composition may consist essentially of barium sulphate and said polymeric material. In other cases, said composition may include one or more other fillers, for example carbon fibre. A composition may include up to 35 wt % or up to 30 wt % carbon fibre. When carbon fibre is included the composition suitably includes 25-35 wt % carbon fibre.

Said barium sulphate may be used to increase the NIIS of said polymeric material by at least 10 KJm⁻², suitably by at least 20 KJm⁻², preferably by at least 30 KJm⁻², more preferably by at least 40 KJm⁻², measured in accordance with ISO180. Said NIIS may be increased by less than 60 KJm⁻².

The ratio of the NIIS of a composition which includes barium sulphate as described to the NIIS of an otherwise identical composition which does not include barium sulphate as described may be at least 2, suitably at least 4, preferably at least 6, more preferably at least 8. The ratio may be less than 15 or less than 11.

The NIIS of a composition comprising said polymeric material and barium sulphate is increased when the composition includes water. Accordingly, the invention extends to the use of barium sulphate and water for increasing the impact strength of a polymeric material as described according to the first aspect.

The ratio of the wt % of water to barium sulphate may be in the range 0.01 to 0.2, preferably in the range 0.02 to 0.15. The ratio is suitably less than 0.10, preferably less than 0.05, more preferably less than 0.04.

The wt % of water in a composition which also includes barium sulphate and said polymeric material may be at least 0.10 wt %, preferably at least 0.2 wt %, more preferably at least 0.30 wt %, especially at least 0.40 wt %. The wt % of water may be less than 1 wt %, less than 0.8 wt %, less than 0.6 wt %. The wt % of water may be in the range 0.3 to 0.7 wt %, suitably 0.4 to 0.6 wt %.

Said composition comprising polymeric material and barium sulphate may be provided in any desired form, for example as a stock shape, film or fibre.

The NIIS of said composition comprising polymeric material and barium sulphate has been found to be reversible in dependence upon whether water is present or not. To benefit from the increase in impact strength it is therefore desirable for a part made from a composition comprising barium sulphate and said polymeric material to be used in a moist and/or wet environment. Thus, the invention extends to the use of barium sulphate for increasing the impact strength of a polymeric material according to the first aspect used in making a part for use in a moist and/or wet environment. Said environment may include water in a liquid state. Water in a liquid state may contact the part in use. Said environment may be substantially enclosed and suitably includes a source of water and/or water vapour. Said environment may comprise a human or animal body (especially the former) and preferably comprises an internal region of a human body. Said part may be an implant.

According to a second aspect of the invention, there is provided a method of increasing the impact strength of a polymeric material as described according to the first aspect, the method comprising forming a composition comprising said polymeric material and barium sulphate.

Said composition, polymeric material, barium sulphate, impact strength and any other features of the invention of the first aspect may be applied to the invention of the second aspect mutatis mutandis.

The method may comprise selecting a said polymeric material and melt compounding it with barium sulphate.

The method may comprise treating the composition to increase the level of water contained therewithin.

According to a third aspect of the invention, there is provided a method of increasing the impact strength of a part which comprises a polymeric material as described according to the first aspect and barium sulphate, the method comprising treating the part to increase the level of water contained therewithin.

The part may have any feature of the composition described according to the first and second aspects after said treatment.

The wt % of water in said part which also includes barium sulphate and said polymeric material may be at least 0.10 wt %, preferably at least 0.20 wt %, more preferably at least 0.30 wt %, especially at least 0.40 wt %. The wt % of water may be less than 1 wt %, less than 0.8 wt %, less than 0.6 wt %. The wt % of water may be in the range 0.3 to 0.7 wt %, suitably 0.4 to 0.6 wt %.

The wt % of barium sulphate in said part may be at least 5 wt %, suitably at least 8 wt %, preferably at least 10 wt %, more preferably at least 13 wt %, especially at least 16 wt %. Said wt % of barium sulphate may be less than 30 wt % or less than 25 wt %.

The wt % of said polymeric material in said part may be at least 40 wt %. In some cases, greater than 99 wt % of said part may consist essentially of barium sulphate and said polymeric material. In other cases, said part may include one or more other fillers, for example carbon fibre. A part may include up to 35 wt % or up to 30 wt % carbon fibre. When carbon fibre is included the part suitably includes 25-35 wt % carbon fibre.

Treatment of said part preferably involves an active treatment. It preferably comprises a treatment at a temperature of greater than 40° C., suitably at greater than 60° C., preferably at greater than 80° C. The part may be treated with steam. The part may be treated under a pressure of greater than ambient pressure, for example greater than 2 bar or 4 bar. Treatment may involve an autoclave and/or may involve steam sterilisation.

The invention extends to a part made in a method of the third aspect.

According to a fourth aspect of the invention, there is provided a part which is made from a composition comprising a polymeric material as described according to the first aspect and barium sulphate.

Preferably said part has been treated to increase its NIIS. The part may have been treated in a method of the second or third aspects

The part is preferably spaced from and/or separated from its position of intended use. The part is preferably outside an environment in which it is intended to be used. For example, if the part is for implantation in a human or animal body (as is preferred) the part may be outside the body but suitably has increased impact strength by virtue of the treatment to which it has been subjected and/or other features of the part described herein.

Said part is preferably an implantable part, for example an implantable prosthesis, such as an orthopaedic implant, for implantation in a human body. It may be a dynamic stabilisation rod or for use in a prosthetic hip joint.

Said part may comprise greater than 50 wt % of said polymeric material (preferably polyetheretherketone), at least 8 wt % (preferably at least 10 wt % or at least 13 wt %) barium sulphate and at least 0.3 wt % (preferably at least 0.45 wt %) of water. In an especially preferred embodiment, said part includes at least 55 wt % polyetheretherketone, at least 10 wt % barium sulphate and at least 0.4 wt % of water. The part may include 0 to 34.6 wt % of other fillers, for example carbon fibre.

According to a fifth aspect of the invention, there is provided a package, which is suitably sterile, comprising a part according to the fourth aspect or a part, the impact strength of which has been increased, in accordance with the third aspect.

Said part may be arranged in a package from which air has been removed. For example, it may be vacuum packed for example in a metal (e.g. aluminium) lined receptacle.

According to a sixth aspect, there is provided the use of a composition comprising a polymeric material according to the first aspect and barium sulphate in the manufacture of an implantable prosthesis for example an orthopaedic implant.

According to a seventh aspect, there is provided a method of making an implantable prosthesis, for example an orthopaedic implant, the method comprising:

(i) selecting a composition comprising a said polymeric material according to the first aspect and barium sulphate;

(ii) melt processing, for example extruding or injection moulding, said composition to define the part or a precursor of said part.

The impact strength of the part may be increased as described according to the second or third aspects.

Any feature of any aspect of any invention or embodiment described herein may be combined with any feature of any aspect of any other invention or embodiment described herein mutatis mutandis.

Specific embodiments of the invention will now be described by way of example.

The following materials are referred to hereinafter.

PEEK-OPTIMA LT1 (Trade Mark)—an implantable grade of polyetheretherketone having an MV of 0.46 KNsm⁻² obtained from Invibio Limited, Thornton, Cleveleys, UK.

Barium Sulphate—Grade 101750, extra pure for X-ray diagnosis.

The influence of barium sulphate on properties of polyetheretherketone was investigated in a range of experiments as described in the following examples.

### EXAMPLE 1

### General Procedure for Preparing Polyetheretherketone/Barium Sulphate Composites

Barium sulphate was added to PEEK-OPTIMA via an extrusion compounding process. By way of example, the barium sulphate can be gravimetrically metered and fed through a side feeder into a twin screw extruder, where it is combined with plasticized polymer melt and intimately mixed to provide a uniform dispersion of the filler within the polymer. Typically the filler may be added up to 60% by weight of the polymer, depending upon the desired mechanical properties of the compound. Extrusion of this mixture through a die generates strands or laces that cool and solidify before being chopped into small granules in preparation for subsequent processing.

### EXAMPLE 2

### General Procedure for Preparing Test Pieces of Composites

The compound prepared in Example 1 was injection moulded to manufacture test pieces, in accordance with the requirements of ISO 527-2 (sample geometry 1B), ISO 178, and ISO 180 for tensile strength, flexural strength and notched Izod impact testing respectively.

### EXAMPLES 3-20

### Preparation and Testing of Test Pieces

Test pieces were prepared as described in Examples 1 and 2 containing PEEK-OPTIMA LT1 and either 4 wt % or 20 wt % of barium sulphate and subjected to a range of different treatments as described in Table 1.

Each test piece was tested in accordance with ISO180 for notched ISOD impact strength. ISO572-2, ISO178 and for tensile properties, flexural properties and ISO180 for notched IZOD impact strength. Results are provided in Table 2.

TABLE 1

| | Amount of Barium Sulphate | |
|---|---|---|
| Pre-Treatment | 4% wt | 20 wt % |
| A) As moulded | Example 3 | Example 12 |
| B) 60 day natural ageing (closed | Example 4 | Example 13 |
| container) | | |
| C) 60 day natural ageing (open | Example 5 | Example 14 |
| to atmosphere) | | |
| D) 60 day natural ageing plus | Example 6 | Example 15 |
| 200kGy gamma | | |
| E) 60 day natural ageing plus | Example 7 | Example 16 |
| 200kGy gamma plus oxygen | | |
| ageing (40 days at 5 bar) | | |
| F) 60 day natural ageing plus | Example 8 | Example 17 |
| 200kGy gamma plus oxygen | | |
| ageing (40 days at 5 bar) plus | | |
| saline soak (3 months at 90° C.) | | |
| to simulate 10 years in-vivo | | |
| G) 60 day natural ageing plus | Example 9 | Example 18 |
| oxygen ageing (40 days at 5 bar) | | |
| plus steam sterilisation (3 cycles) | | |
| plus saline soak (3 months at | | |
| 90° C.) | | |
| H) 60 day natural ageing plus 3 | Example 10 | Example 19 |
| steam cycles (134° C.) | | |
| I) 60 days natural ageing plus 3 | Example 11 | Example 20 |
| EtO cycles | | |

TABLE 2

| Example No | Notched Izod Impact KJ/m² |
|---|---|
| 3 | 7.97 |
| 4 | 7.2 |
| 5 | 10.71 |
| 6 | 11.73 |
| 7 | 7.86 |
| 8 | 11.63 |
| 9 | 9.01 |
| 10 | 9.7 |
| 11 | 10.58 |
| 12 | 7.7 |
| 13 | 6.13 |
| 14 | 19.15 |
| 15 | 20.36 |
| 16 | 16 |
| 17 | 48.03 |
| 18 | 26.34 |
| 19 | 53.99 |
| 20 | 20.76 |

### EXAMPLES 21-25

### Comparison of Dried and “Wet” Test Pieces

Test pieces were prepared as described in Example 2 with different filler loadings, varying between 4 to 20 wt % and the Notched Izod Impact Strength assessed in accordance with ISO180 under two different sets of conditions—firstly, after drying in an oven at 120° C. for 72 hours; and, secondly, after sterilisation in steam for 20 minutes at 134° C., soaking in water for 15 hours followed by further sterilisation in steam for 20 minutes at 134° C. Results are provided in Table 3.

TABLE 3

| | Amount of barium | Notched Izod Impact KJ/m² | |
|---|---|---|---|
| | sulphate | | After sterilisation/ |
| Example No | wt % | After Drying | soaking/sterilisation |
| 21 | 4 | 5.08 | 5.6 |
| 22 | 6 | 4.36 | 6.75 |
| 23 | 10 | 3.82 | 7.58 |
| 24 | 15 | 3.74 | 8.6 |
| 25 | 20 | 4.17 | 16.54 |

### EXAMPLE 26

### Cyclical Testing

Respective test pieces comprising 4 wt % and 20 wt % barium sulphate were prepared and tested in cyclical testing.

Firstly, a 4 wt % barium sulphate test piece was tested after moulding; then it was dried at 120° C. for 72 hours and re-tested; thereafter it was sterilised in steam for 20 minutes at 134° C. and re-tested; and finally it was re-dried and re-tested. Results are provided in Table 4.

TABLE 4

| | Amount barium | Time when test | Notched Izod |
|---|---|---|---|
| Example No | sulphate (wt %) | undertaken | Impact KJ/m² |
| 26 | 4 | After moulding | 7.97 |
| 27 | 4 | After initial drying | 6.74 |
| 28 | 4 | After sterilization | 9.64 |
| 29 | 4 | After re-drying | 4.64 |
| 30 | 20 | After moulding | 7.7 |
| 31 | 20 | After initial drying | 5.37 |
| 32 | 20 | After sterilization | 29.8 |
| 33 | 20 | After re-drying | 5.79 |

### Discussion

The effect on impact strength of the incorporation of barium sulphate and water into polyaryletherketone may have many industrial applications. It may be of particular utility for making parts which are used in a moist environment so that the optimum amount of water (believed to be about 0.15 wt %) may be maintained within a composition which comprises the polyaryletherketone and barium sulphate. A suitably moist environment is found in the human body and, consequently, parts made from polyaryletherketone and barium sulphate and, optionally, other fillers, may be pre-conditioned so they contain water, for example 0.15 wt %, and then implanted. When implanted, the water level is maintained and so, therefore, is the surprising improvement in impact strength. It is also found that other properties, for example other mechanical properties of the polyaryletherketone are not significantly detrimentally affected by incorporation of barium sulphate and/or water. Furthermore the polyaryletherketone is chemically unchanged.

1. A method of increasing the impact strength of a polymeric material which has a moiety of formula 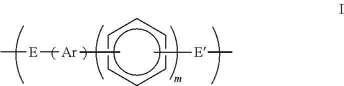
and/or a moiety of formula 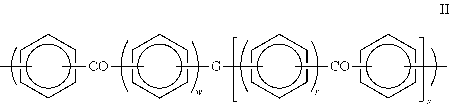
and/or a moiety of formula 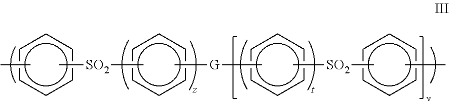
wherein m, r, s, t, v, w and z independently represent zero or a positive integer, E and E′ independently represent an oxygen or a sulphur atom or a direct link, G represents an oxygen or sulphur atom, a direct link or a —O—Ph—O— moiety where Ph represents a phenyl group and Ar is selected from one of the following moieties (i)**, (i) to (iv) which is bonded via one or more of its phenyl moieties to adjacent moieties 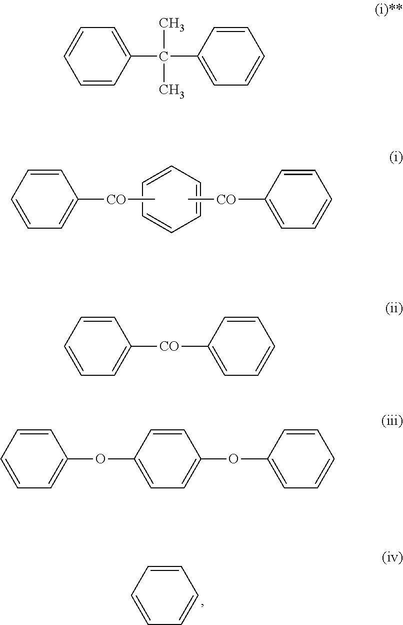
the method comprising forming a composition comprising said polymeric material and barium sulphate.

2. The method according to claim 1, wherein said polymeric material comprises a repeat unit of formula 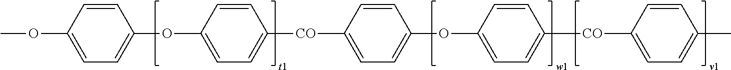
where t1, and w1 independently represent 0 or 1 and v1 represents 0, 1 or 2.

3. The method according to claim 1, wherein c1=1, v1=0 and w1=0.

4. The method according to claim 1, wherein said polymeric material has a Notched Izod Impact Strength (specimen 80 mm×10 mm×4 mm with a cut 0.25 mm notch (Type A), tested at 23° C., in accordance with ISO180) of at least 4 KJm⁻².

5. The method according to claim 1, wherein said barium sulphate has a D₁₀ particle size in the range 0.1 to 1.0 μm; a D₅₀ particle size in the range 0.5 μm to 2 μm and a D₉₀ particle size in the range 1.0 μm to 5 μm.

6. The method according to claim 1, wherein the ratio of the wt % of barium sulphate to the wt % of said polymeric material is greater than 0.04 and is less than 0.4.

7. The method according to claim 1, wherein the wt % of barium sulphate in a composition which includes barium sulphate and said polymeric material is at least 10 wt %.

8. The method according to claim 1, wherein said composition comprising said polymeric material and barium sulphate has a Notched Izod Impact Strength that is increased by at least 10 KJm⁻².

9. The method according to claim 1, comprising forming a composition comprising said polymeric material, barium sulphate and water.

10. The method according to claim 9, wherein the ratio of the wt % of water to barium sulphate is in the range 0.01 to 0.2.

11. The method according to claim 9, wherein the wt % of water in a composition which includes barium sulphate and said polymeric material is less than 1 wt %.

12. (canceled)

13. (canceled)

14. A method according to claim 1, which comprises treating the composition to increase the level of water contained therewithin.

15. A method of increasing the impact strength of a part which comprises a polymeric material as defined in claim 1 and barium sulphate, the method comprising treating the part to increase the level of water contained therewithin.

16. A method according to claim 15, wherein the wt % of water in said part which includes barium sulphate and said polymeric material is at least 0.10 wt % and is less than 1 wt %; the wt % of barium sulphate in said part is at least 10 wt % and is less than 30 wt %; and the wt % of said polymeric material in said part is at least 40 wt %.

17. A method according to claim 15, wherein treatment of said part involves an active treatment.

18. A part made in a method according to claim 15.

19. A part which comprises a polymeric material as defined in claim 1, barium sulphate and water.

20. A part according to claim 19, which is an implantable prosthesis.

21. A package comprising a part according to claim 18.

22. (canceled)

23. A method of making an implantable prosthesis, the method comprising
(i) selecting a composition comprising a polymeric material as defined in claim 1 and barium sulphate;
(ii) melt processing said composition to define the part or a precursor of said part.

24. A part according to claim 19, wherein the part is designed for use in a moist and/or wet environment.
